(19) United States
(12) Patent Application Publication
(10) Pub. No.: US 2014/0154344 A1
(43) Pub. Date: Jun. 5, 2014

(54) AUTISM IMPROVING AGENT AND AUTISM IMPROVING TEA

(71) Applicant: Michio TANI, Meguro-ku, Tokyo (JP)

(72) Inventor: Michio Tani, Tokyo (JP)

(21) Appl. No.: 14/122,631

(22) PCT Filed: May 26, 2011
(86) PCT No.: PCT/JP2011/002951

§ 371 Date: Feb. 18, 2014

(51) Int. Cl. A61K 36/752; A61K 36/23; A61K 31/70; A61K 36/48; A61K 36/53; A61K 36/899; A61K 36/534

(52) U.S. Cl. CPC A61K 36/752; A61K 36/899; A61K 36/23; A61K 36/534; A61K 36/48; A61K 36/53; A61K 31/70
USPC 424/736

(57) ABSTRACT

Provided is an improving agent that is more suitable to improvement of autism, the agent having reduced side effects thanks to the suppression of the immunological factors. An autism improving agent is provided that is characterized in that the inventive agent contains celery seed, wheat, lemon balm, mint, fenugreek seed, stevia, laurel, brown sugar powder and rosemary and that the agent has an effect of improving the symptoms relative to autism.

### TECHNICAL FIELD

The present invention relates to an autism improving agent, autism improving tea and a producing method thereof, and in particular, to a more effective autism improving agent and autism improving tea.

### BACKGROUND ART

Autism is a development disorder that is indicative of a wide range of symptoms that retard the development of the social ability, in particular, the ability to communicate with others. Such symptoms cover a large extent of the disorder in a non-step manner: starting from those without involving any intellectual disorder, and up to those involving severe intellectual disorder. Hence, they are often called autism spectrum disabilities. The symptoms are regarded as a pervasive developmental disorder in Diagnostic and Statistical Manual of Mental Disorders (hereinafter, DSM) issued by the American Psychiatric Association. The basic feature of the pervasive developmental disorder is that the qualitative disorder in human relationship, the qualitative disorder in communication, and the stereotypical behaviors, activities and interests are obviously disproportionate for their development level and their mental ages. The incidence rate, which was considered 4 or 5 out of every 10 thousand people, is increasing, and a recent report shows that the rate is now 1 out of every 150 people (Non-patent Document 1). No effective therapy has been discovered yet.

In the past, autism was regarded as a psychosomatic disorder, but actually, the etiology of autism is still unknown. Autism is now considered to be generated by the involvement of genetic diathesis, and a report shows the high incidence of autism for identical twins and the high risk of familial occurrence (Non-patent Document 2).

Autism a pervasive developmental disorders (a series of disorders characterized by the qualitative disorder in the patterns of interactive social relationships and communication, as well as by the localized stereotyped repetitive interest and range of behaviors) that is defined by the abnormal functions having the specific types for all of the three zones of: abnormality and/or disorder of development; interactive social relationships and communication; and localized repetitive behaviors.

These types of symptoms are sometimes not called autism but a developmental disorder or behavioral disorder characterized by the symptom of hyperactivity, negligence and impulsiveness.

In contrast, the number of reports in the medical world is recently on the increase that suggests the influence of environmental factors such as chemical substance and foods or beverages (Non-patent Document 3). Vargas et al. reported that astrocyte and microglia remarkably activate around the cerebellum, which involves the production of MCP-I and TGF-Beta 1 that damage the tissue. Vargas et al. also suggested the existence of inflammation of the central nerve (Non-patent Document 4). Abnormalities in the antibody level, in cytokine, and in cell factor were also reported, and the connection with autoimmune disease was also pointed out. Thus, a variety of immunological factors has been suggested (Non-patent Documents 5 to 14).

As to this environmental factor, Non-patent Document 15 has the following statement: The number of autistic children has increased 7 to 8 times in the State of California from 1990 on. This rapid increase cannot be ascribed only to inclusion of the lighter cases, inclusion of younger children to the subject of diagnosis, and the demographics, but should also be ascribed to other factors including the environmental factors. In other words, the traditional theory of the congenital brain function disorder that is based solely on the traditional genital factors has been overturned. It is now required to take into account the possibility that the environmental factors may be the cause of autism both inherently and acquiredly. Accordingly, elimination of such harmful environmental factors such that the autistic children will regain their healthy conditions has become a real possibility.

Even before the environmental factor theory, some suggestions were rendered that the autism was a brain function disorder and that some attempt should be made to improve the brain function by oral administration of drugs. For example, Patent Document 1 suggests an autism improving agent containing, as the effective component, 2-(4-methylaminobutoxy)diphenylmethane, its hydrate, or a salt thereof that is pharmacologically permissible.

In contrast, Patent Document 2 proposes an improving agent for autism using Chinese parsley.

In Patent Document 3, the Applicants propose an autism improving agent using a dry substance of celery seed, lemon balm, fenugreek seed, lemongrass, mint and stevia, and a hydrothermal extract thereof.

### CITATION LIST

### Patent Literature

PTL 1: Japanese Laid-open Patent Publication No. H08-119868
PTL 2: Japanese Laid-open Patent Publication No. 2006-199662
PTL 3: Japanese Laid-open Patent Publication No. 2011-046659

### Non Patent Literature

NPL 1: Fombonne E. et al.: Epidemiology of Pervasive Developmental Disorders, Pediatr Res., Vol. 65, 591-8, 2009
NPL 2: Bailey A et al: Autism as a Strongly Genetic Disorder: Evidence from a British Twin Study, Psychol Med, Vol. 25, 63-77, 1995
NPL 3: Curtis L T et al.: Nutritional and Environmental Approaches to Preventing and Treating Autism and Attention Deficit Hyperactivity Disorder (ADHD), a Review J Altern Complement Med., Vol. 14, 79
NPL 4: Vargas D L et al.: Neuroglial Activation and Neuroinflammation in the Brain of Patients with Autism, Ann Neurol, Vol. 57, 67-81, 2005
NPL 5: Zimmerman et al.: Cerabrospinal Fluid and Serum Markers of Inflammation in Autism, Pediatr Neurol, Vol. 33, 198-205, 2006
NPL 6: Molloy C A et al.: Elevated Cytokines Levels in Children with Austin Spectrum Disorder, J Neurommunol, Vol. 172, 198-205, 2006
NPL 7: Singer H S et al.: Antibrain Antibodies in Children with Autism and their Un-affected Siblings, J Neurommunol, Vol. 178, 149-155, 2006
NPL 8: Braunschweig D et al.: Autiam: Maternally Derived Antibodies Specific for Fetal Brain Proteine, Neurotoxicology, Vol. 29, 226-231, 2008
NPL 9: Cohly H H et al.: Immunological Findings in Autism, Neurobiol, Vol. 71, 817-841, 2005
NPL 10: Sweeten T L et al.: Increased Prevalence of Familial Autoimmunity in Probands with Pervasive Developmental Disorders, Pediatrics, Vol. 112, e420, 2003
NPL 11: Comi A M et al.: Familial Clustering of Autoimmune Disorders and Evaluation of Medical Risk Factors in Autism, J Child Neurol, Vol. 14, 388-394, 1999
NPL 12: Ashwood P et al.: The Immune Response in Autism: a New Frontier for Autism Research, J Leukoc Biol, Vol. 80, 1-15, 2006
NPL 13: Sweeten T L et al., High Nitric Oxide Production in Autistic Disorder: a Possible Role for Interferon-gamma, Biol Psychiatry, Vol. 55, 434-437
NPL 14: Warren R P et al., Reduced Natural Killer Cell Activity in Autism, J Am Acad Child Adolesc Psychiatry, Vol. 26, 333-5, 1987
NPL 15: Surging Autism Incidence in California, Medical Tribune, Apr. 2, 2009, p. 32

### SUMMARY OF INVENTION

### Technical Problem

However, an improving agent using 2-(4-methylaminobutoxy)diphenylmethane cannot completely eliminate the side effect. Another problem is that an improving agent using Chinese parsley has a smell which cannot be completely eliminated, and that the improving effect thereof was not clear.

In addition, the autism improving agent developed by the Applicant as disclosed in Patent Document 3 could not necessarily obtain the maximum improvement.

Besides, no obvious achievements have been established that inhibit the symptom by way of suppressing the immunological factors.

The present invention is intended to solve the aforementioned problems. It is thus the object of the present invention to provide an improving agent that is more suitable to improvement of autism, the agent having reduced side effects thanks to the suppression of the immunological factors.

### Solution to Problem

The inventor of the present invention has been diligently committed to research the component having better advantageous effect in comparison with the autism improving agent disclosed in Patent Document 3 through his 40-year clinical experience of Chinese herbal medicine, and found that the object could be achieved by means of wheat, laurel, brown sugar powder and rosemary in place of lemongrass.

The autism improving agent according to the embodiment of the present invention is characterized in that the inventive agent contains celery seed, wheat, lemon balm, mint, fenugreek seed, stevia, laurel, brown sugar powder and rosemary and that the agent has an effect of improving the symptoms relative to autism.

The inventive agent may contain 2 to 8 grams of celery seed, 0.5 to 4 grams of wheat, 2 to 8 grams of lemon balm, 0.5 to 3 grams of mint, 1 to 6 grams of fenugreek seed, 0.5 to 4 grams of stevia, 1 to 6 grams of laurel, 0.1 to 2 grams of brown sugar powder, and 1 to 6 grams of rosemary. It is to be noted that the above amount of each component is for adults, and the suitable amount for children is one-third to two-thirds of the amount for adults.

The autism improving tea according to the embodiment of the present invention is characterized in that the inventive agent contains celery seed, wheat, lemon balm, mint, fenugreek seed, stevia, laurel, brown sugar powder and rosemary and that the agent has an effect of improving the symptoms relative to autism.

The autism improving tea is characterized in that it contains 2 to 8 grams of celery seed, 0.5 to 4 grams of wheat, 2 to 8 grams of lemon balm, 0.5 to 3 grams of mint, 1 to 6 grams of fenugreek seed, 0.5 to 4 grams of stevia, 1 to 6 grams of laurel, 0.1 to 2 grams of brown sugar powder, and 1 to 6 grams of rosemary. It is to be noted that the above amount of each component is for adults, and the suitable amount for children is one-third to two-thirds of the amount for adults.

### Advantageous Effects of Invention

The present invention enables provision of an improving agent suitable for autism having reduced side effects thanks to the suppression of pathological immunological factors.

### BRIEF DESCRIPTION OF DRAWINGS

FIG. 1a is an exemplary view of an interview sheet to check the degree of autism.

FIG. 1b is another exemplary view of an interview sheet to check the degree of autism.

FIG. 2 is indicative of the transition of the CARS average total scores of patients taking the autism improving agent or the autism improving tea according to the present invention.

### DESCRIPTION OF EMBODIMENTS

Embodiments of the present invention are described below in detail with reference to the drawings.

The improving agent suitable for autism according to the present invention contains celery seed, wheat, lemon balm, mint, fenugreek seed, stevia, laurel, brown sugar powder and rosemary. The suitable amount of each component of the agent is preferably 2 to 8 grams of celery seed, 0.5 to 4 grams of wheat, 2 to 8 grams of lemon balm, 0.5 to 3 grams of mint, 1 to 6 grams of fenugreek seed, 0.5 to 4 grams of stevia, 1 to 6 grams of laurel, 0.1 to 2 grams of brown sugar powder, and 1 to 6 grams of rosemary.

Celery seed (academic name: Apium graveolens L.), which is an ingredient for the present invention, is the seed of a plant classified into Apiaceae, Apiales. The seed of the celery has traditionally been used as dried spice since ancient Egypt for edible or medicinal purposes, and hence does not pose any safety problem.

Wheat, which is an ingredient for the present invention, is an annual grass classified into Triticum, Poaceae, and the seed thereof is ground to be used as flour. The component of flour comprises: starch which is a carbohydrate; gliadin and glutenin as protein; linoleic acid and linolenic acid and the like for fat; vitamins B1, B2, E, pantothenic acid and niacin for vitamins; and phosphorus, calcium, iron, potassium, sodium, magnesium and the like for minerals. Wheat has been used for edible purposes since before Christ, and hence does not pose any safety problem.

Lemon balm, which is an ingredient for the present invention, is a perennial plant classified into Lamiaceae, and the leaf thereof has a characteristic of the smell like lemon. Lemon balm contains citral and citronellol as active ingredient. A positive effect of lemon balm is an anti-allergy effect. Other positive effects include: active oxygen removal effect; digestion promotion effect; tranquilizing effect; detoxication effect; antidepressant effect; cephalalgia relaxation effect; alergy reaction relaxation effect such as pollen alergy. Besides, lemon balm is advantagous for direct application to skin in the form of essential oil for improvement of skin troubles such as eczema, and for insect deterrent and hemostasis. Lemon balm has been used for edible purposes, and hence does not pose any safety problem.

Mint (academic name: Mentha arvensis L.), which is an ingredient for the present invention, is a plant classified into Lamiaceae. The essential oil of mint, rich in menthol, has traditionally been applied for fragrance use since ancient Rome. Mint has been used for edible purposes, and hence does not pose any safety problem.

Fenugreek seed, which is an ingredient for the present invention, is a seed of a plant (academic name: Trigonella foenum-graecum L.) classified into Trigonella, Fabaceae. The dried seed of Fenugreek is a spice having sweet smell like caramel. Fenugreek seed has been widely used for edible or medicinal purposes since ancient Egypt, and hence does not pose any safety problem.

Stevia, which is an ingredient for the present invention, is Stevia rebaudiana Bertoni, a perennial plant originated from South America, and classified into Asteraceae. Stevia has been generally used as a sweetener and the like, and hence does not pose any safety problem.

Laulus, which is an ingredient for the present invention, is Laulus nobilis L., an evergreen small-tall tree originated from the coastal area of the Mediterranean Sea, and classified into Lauraceae. Laurel contains essential oil components of terepenes such as 1,8-cineol (30-70%), linalol, methyl eugenol and pinene (having the basic skeleton of carbon number 10) and sesquiterpenes such as costunolide (having the basic skeleton of carbon number 15). Laurel has been widely used for edible or medicinal purposes, and hence does not pose any safety problem.

Brown sugar powder, which is an ingredient for the present invention, is a blackish-brown sugar produced by boiling down the sugarcane juice. The component mainly comprises sucrose, and also contains calcium, phosphorous, iron, sodium, potassium, vitamin B1, vitamin B2, vitamin B3, and vitamin B6. Brown sugar powder has been widely used for edible or medicinal purposes, and hence does not pose any safety problem.

Rosemary, which is an ingredient for the present invention, is a herb and is an evergreen low tree (academic name: Rosmarinus officinalis L.), classified into Rosmarinus, Lamiaceae. The raw or dried leaf of Rosemary has been widely used for edible or medicinal purposes since ancient Greece or Rome, and hence, use of rosemary does not pose any safety problem with the exception of the essential oil thereof.

Examples of the formulation of the inventive autism improving agent include an internal drug such as tablet, capsule, powdered formulation, suspension agent, and liquid formulation. Besides, such formulation can also be used in the form of an injectable solution.

The above-mentioned products can be produced in the conventional method. One can add, if necessary, diluents, adjuvants, additives and the like to produce an internal drug. Diluents are classified into filler, extender and the like; to be more specific, classified into sugar, starch, inorganic substance, crystalline cellulose and the like. Adjuvants are classified into buffering agent, emulsifier, dispersant, binder, lubricant, disintegrant and the like. Additives are classified into preserving agent, aromatic substance, flavoring agent and the like.

A method of using the inventive autism improving agent includes an oral administration method by way of powdered formulation, granular formulation, capsule formulation and the like. Other administration method can include subcutaneous injection, intramuscular injection, intravenous infusion and the like.

Dosage may vary depending on the symptom or the formulation, but generally speaking, dosage is preferably within the range of 20 mg to 500 mg per day of active ingredient.

### EXAMPLE

The present invention will now be explained in detail according to the Examples.

A therapy using the autism improving agent according to the present invention was conducted for three autistic children cases: (A) 30 months after the first medical examination; (B) 12 months after the first medical examination; and (C) 6 months after the first medical examination. All cases being male).

The autism improving agent according to the present invention can take a form of hydrothermal extract of celery seed, wheat, lemon balm, mint, fenugreek seed, stevia, laurel, brown sugar powder and rosemary. In this case, hydrothermal extraction of these ingredients in a raw condition is also possible, but hydrothermal extraction of these ingredients in a dried condition is more preferable. Still more preferably, the dried ingredients are further roasted and then hydrothermal-extracted. The hydrothermal extract solution of the roasted and hydrothermal-extracted ingredients can also be spray-dried or free-dried to make extract powder, granules, tablets and the like for effective medication use. Besides, the biological therapeutic substance of the present invention can be directly taken as a dried or roasted ground product without hydrothermal extraction.

During the hydrothermal extraction, the above-mentioned dried or roasted substance can be directly hydrothermally extracted, but from the practical point of view, it is more preferable that the substance is further finely ground and then is extracted. Besides, the weight ratio between the hot water to be used as extract solvent and the above-mentioned dried or roasted substance is not particularly specified; but preferably, the ratio is 10 to 80 weight times of hot water in comparison with the above-mentioned dried or roasted substance, in particular, 20 to 50 weight number of hot weight in consideration of the operation and the efficiency of the extraction. The extraction is efficiently conducted at an elevated temperature, but can also be sufficiently conducted even at a low temperature. 70 to 100 Celsius are preferable. The extraction time can be set long or short so long as the active ingredients are sufficiently extracted, and can be properly determined in consideration of the extraction temperature and the amount of the extracted hot water. Besides, the extraction can be conducted under increased pressure, ordinary pressure, or reduced pressure. Most preferable extraction condition is under the ordinary pressure, within the extraction temperature range of 85 to 95 Celsius, and within the extraction time of 3 to 6 minutes.

With regard to the roasting method, the dried ingredients of celery seed, wheat, lemon balm, mint, fenugreek seed, stevia, laurel, brown sugar powder and rosemary can be directly roasted; but from the practical point of view, the dried ingredients are preferably roughly ground and then are roasted. Rough grinding of the aforementioned dried ingredients requires using a grinder and the like to grind them into pieces of 0.2 to 3.0 mm by way of the conventional grinding method. Besides, the roasting method can take any conventional roasting method such as sand roasting, wire mesh roasting, hot-air roasting, and microwave oven roasting. The roasting conditions such as roasting time and temperature can be properly determined in consideration of the amount of the ingredients to be roasted at one roasting. For example, the roasting temperature and time for roasting 100 gram mixture are preferably 110 to 130 Celsius, and 10 to 18 minutes, respectively.

The autism improving agent according to the present application contains celery seed, wheat, lemon balm, mint, fenugreek seed, stevia, laurel, brown sugar powder and rosemary. The suitable amount of each component of the agent is preferably 2 to 8 grams of celery seed, 0.5 to 4 grams of wheat, 2 to 8 grams of lemon balm, 0.5 to 3 grams of mint, 1 to 6 grams of fenugreek seed, 0.5 to 4 grams of stevia, 1 to 6 grams of laurel, 0.1 to 2 grams of brown sugar powder, and 1 to 6 grams of rosemary.

The intake manners of the inventive autism improving agent are not specifically limited.

The intake amount of the autism improving agent according to the present application can be properly determined depending on a variety of conditions such as gender, weight, age, type and extent of the disease, formulation, administration route, and number of doses. In the case of oral administration, the suitable amount of the dried ingredients is typically 9 to 30 grams per day for intake by way of hydrothermal extraction, or for intake by way of hydrothermal extraction after roasting. In particular, extraction of the ingredients packed in teabags made from a material such as paper will allow for easier disposal. For intake by way of extract powder, granule, tablet and the like after spray-drying or freeze-drying the hydrothermal extract solution, the intake amount is preferably 4.0 to 7.0 grams including some proper quantity of diluents.

Next, the autism improving agent according to the present application can be blended into, for example, common foods such as soups, beverages (juice, sake, mineral water, coffee, tea and the like), confectioneries (gum, candy, chocolate, snack, jelly and the like), noodles (soba, udon, ramen and the like), and health foods and nutritional supplements (nutrition-supplement drink and the like). This enables smoother consumption of the autism improving agent in daily life.

Besides, the concentration of biological treatment material in the food or beverage according to the present invention can be properly changed depending on the type of the food or beverage. When the extract powder that is produced by spray-drying or freeze-drying the hydrothermal extract solution is mixed, some diluents may as well be blended such that the daily intake will be 2.0 to 12.0 grams, more preferably 4.0 to 7.0 grams. Meanwhile, the aforementioned concentration is a mere example, which can be properly changed depending on a variety of conditions.

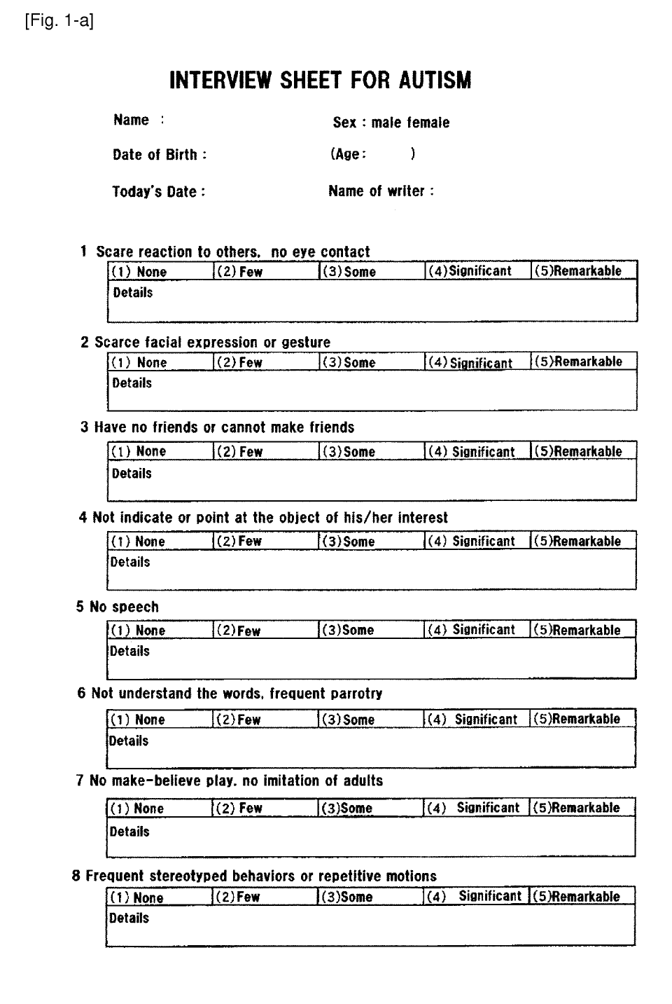

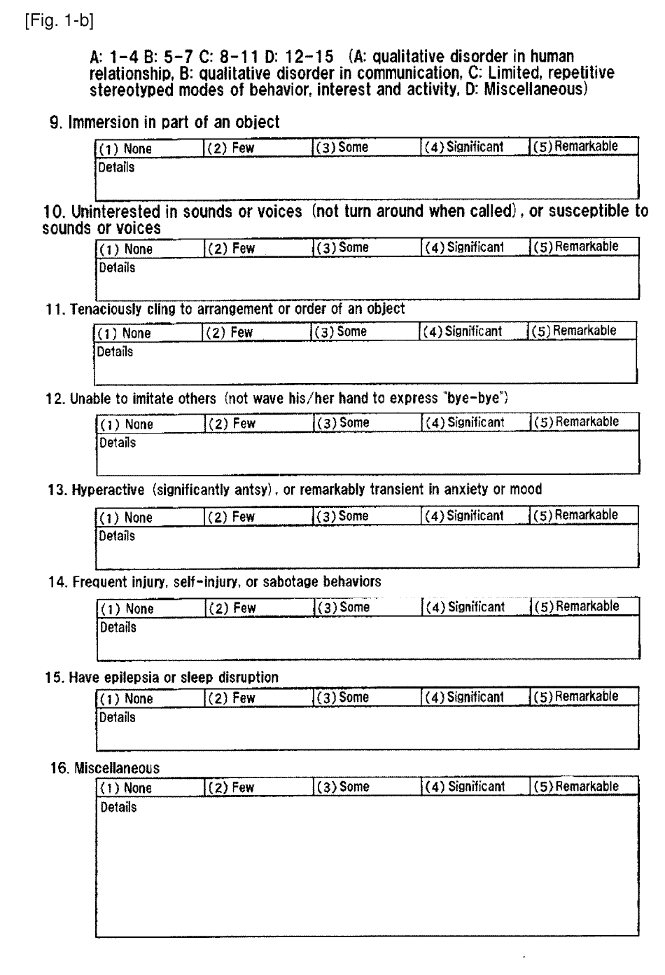

Evaluation was conducted on the basis of the CARS (Childhood Autism Rating Scale) as shown in FIGS. 1a and 1b, and comparison of the symptoms was rendered between before the start of the first treatment, after 6 months, after 12 months, and after 18 months.

The CARS (Childhood Autism Rating Scale) is an evaluation scale for diagnosing the autism and the severity of the symptoms by calculating the sum of the score of the 15 items of behavioral evaluation, and is employed in TEACCH (Treatment and Education of Autistic and related Communication handicapped Children), which is an autism treatment education program in the U.S.A.

Following is the list of the 15 items of behavioral evaluation. 1. Relationship to people: for evaluating the behavior in a situation when he/she faces some human contact. 2. Imitation: for evaluating the verbal ability, behavioral ability as well as imitation ability. 3. Emotional response: for evaluating whether emotional response suitable to the situation is observed. 4. Body use: for evaluating whether he/she can use his/her body for his/her age, and the coordination and suitableness of the body action. 5. Object use: for evaluating the interest in objects, and whether he/she suitably uses the objects. 6. Adaptation to change: for evaluating whether he/she can respond to difficulties in routine and pattern variability and to changes. 7. Visual response: for evaluating whether he/she looks at a person or an object; whether he/she does/does not take a curious look such as gazing at thin air. 8. Listening response: for evaluating how he/she reacts to sounds or words; whether he/she is susceptible or uninterested. 9. Taste-smell-touch response and use: for evaluating whether the response in taste sense, olfactory sense, tactile sense is normal. 10. Fear and nervousness: for evaluating the abnormal fear or incomprehensible fear. 11. Verbal communication: for evaluating whether with or without speech, with or without parrotry or strange way of speaking. 12. Non-verbal communication: for evaluating whether with or without any reaction or expression to a facial expression or to a gesture. 13. Activity level: for evaluating whether he/she is hyperactive or akinesia, and whether he/she can suppress his/her behavior. 14. Level and consistency of intellectual response: for evaluating whether or not he/she has retardation or unbalance in his/her intellectual function. 15. General impressions: for conducting general evaluation of the degree of autism based on the subjective impressions of the examiner.

Each of the 15 items has the following scores: (1) normal: 1 to 1.5 points; (2) slightly abnormal: 2 to 2.5 points; (3) moderately abnormal: 3 to 3.5 points; (4) severely abnormal: 4 points. The examinee having the total score of 30 or higher is diagnosed to be autism, although some Japanese report asserts that the threshold score should be 26. The inventor studied whether there is any significant difference between the points of time: before the start of the first treatment, after 8-month, after 12-month, and after 18-month, with regard to each of the 15 items at each point of time and to the average value of the sum of each of the 15 items by way of the t-test (paired t-test).

Evaluation of the case (A) showed that the average of the sum of each of the 15 items was 42.0 points before the start of the first treatment, 34.5 points after 6 months, 28.0 points after 12 months, below 26.0 points after 18 months, and 24.0 points after 24 months, indicating a significant improvement. The score is below the threshold value after 18 months from the start of the treatment.

Evaluation of the case (B) showed that the average of the sum of each of the 15 items was 38.0 points at the start of the first treatment, 32.0 points after 6 months, and 28.0 points after 12 month, indicating a significant improvement.

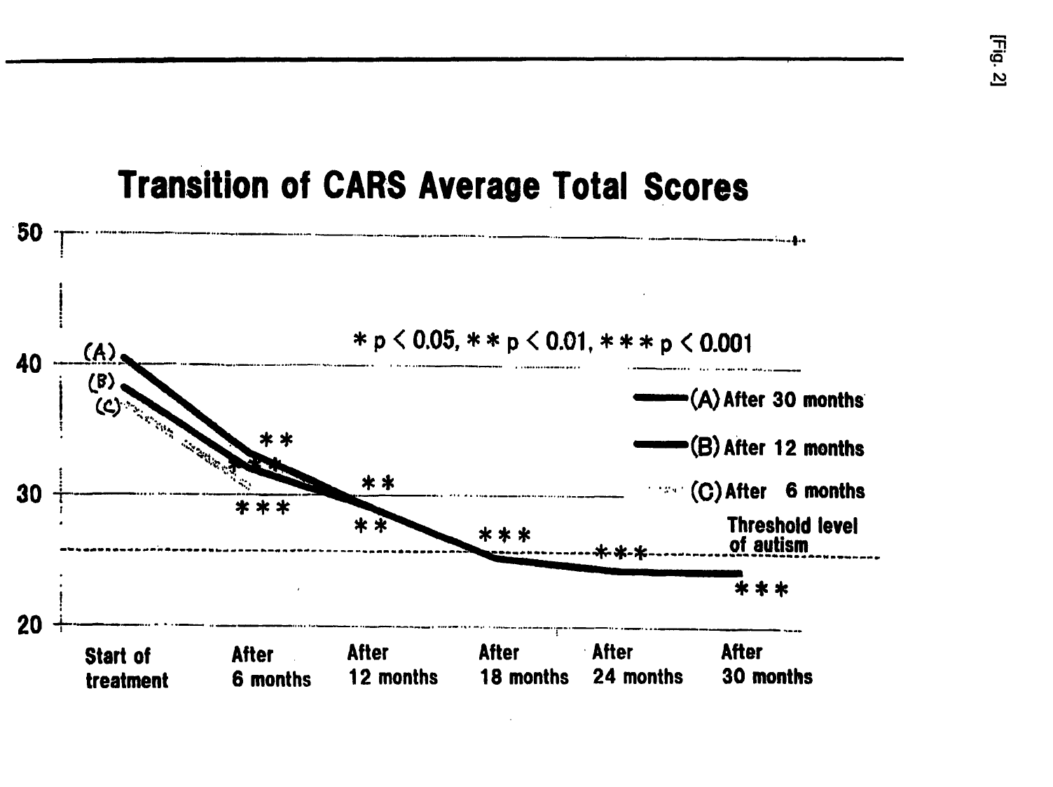

Evaluation of the case (C) showed that the average of the sum of each of the 15 items was 37.0 points at the start of the first treatment, and 30.5 points after 6 months, indicating a significant improvement (FIG. 2).

Consequently, it is clearly shown that application of the autism improving agent according to the present invention for 18 months or longer enables improvement of the symptoms to be below the threshold level of autism.

1. An autism improving agent characterized in that the inventive agent contains celery seed, wheat, lemon balm, mint, fenugreek seed, stevia, laurel, brown sugar powder and rosemary and that the agent has an effect of improving the symptoms relative to autism.

2. The autism improving agent according to claim 1, characterized in that it contains 2 to 8 grams of celery seed, 0.5 to 4 grams of wheat, 2 to 8 grams of lemon balm, 0.5 to 3 grams of mint, 1 to 6 grams of fenugreek seed, 0.5 to 4 grams of stevia, 1 to 6 grams of laurel, 0.1 to 2 grams of brown sugar powder, and 1 to 6 grams of rosemary.

3. An autism improving tea characterized in that the inventive agent contains celery seed, wheat, lemon balm, mint, fenugreek seed, stevia, laurel, brown sugar powder and rosemary and that the agent has an effect of improving the symptoms relative to autism.

4. The autism improving tea according to claim 3, characterized in that it contains 2 to 8 grams of celery seed, 0.5 to 4 grams of wheat, 2 to 8 grams of lemon balm, 0.5 to 3 grams of mint, 1 to 6 grams of fenugreek seed, 0.5 to 4 grams of stevia, 1 to 6 grams of laurel, 0.1 to 2 grams of brown sugar powder, and 1 to 6 grams of rosemary.
